(19) United States
(12) Patent Application Publication
(10) Pub. No.: US 2018/0000328 A1
(43) Pub. Date: Jan. 4, 2018

(54) CAPSULE ENDOSCOPE

(71) Applicant: OLYMPUS CORPORATION, Tokyo (JP)

(72) Inventor: Takuto IKAI, Tokyo (JP)

(73) Assignee: OLYMPUS CORPORATION, Tokyo (JP)

(21) Appl. No.: 15/708,481

(22) Filed: Sep. 19, 2017

Related U.S. Application Data
(63) Continuation of application No. PCT/JP2016/054455, filed on Feb. 16, 2016.

(30) Foreign Application Priority Data
Jul. 31, 2015 (JP) 2015-152114

(51) Int. Cl. A61B 1/04; H04N 5/225; A61B 1/00; A61B 1/06

(52) U.S. Cl. CPC A61B 1/041; A61B 1/06; H04N 5/2256; A61B 1/00016; A61B 1/00027; H04N 2005/2255

(57) ABSTRACT

A capsule endoscope configured to be introduced into a subject is provided. The capsule endoscope includes: a magnetic switch configured to operate depending on a magnetic field externally applied; a first device including an inductor and configured to execute a predetermined function; a second device including no inductor and configured to execute a function different from that of the first device; and a controller configured to drive the second device without driving the first device when the magnetic field is applied to the magnetic switch.

### CROSS-REFERENCE TO RELATED APPLICATION

This application is a continuation of PCT International Application No. PCT/JP2016/054455 filed on Feb. 16, 2016 which claims the benefit of priority from Japanese Patent Application No. 2015-152114 filed on Jul. 31, 2015, the entire contents of which are incorporated herein by reference.

### BACKGROUND

### 1. Technical Field

The disclosure relates to a capsule endoscope which is introduced into a subject to acquire an in-vivo image of the subject.

### 2. Related Art

Conventionally, a capsule endoscope is known which is orally introduced into a subject to capture an image of the inside of the subject, and wirelessly transmits obtained image information to an external device disposed outside the subject. Such a capsule endoscope includes a boosting circuit using a cored coil. The boosting circuit boosts a voltage of power supplied from a power supply to a voltage suitable for an illumination unit disposed inside the capsule endoscope and supplies the power (see JP 2007-222641 A and WO 2010/071075 A).

The capsule endoscope includes therein a magnetic switch whose connection state changes depending on the presence or absence of a magnetic field. Activation of the capsule endoscope is achieved by bringing a starter for applying a magnetic field close to the capsule endoscope.

### SUMMARY

In some embodiments, a capsule endoscope configured to be introduced into a subject is provided. The capsule endoscope includes: a magnetic switch configured to operate depending on a magnetic field externally applied; a first device including an inductor and configured to execute a predetermined function; a second device including no inductor and configured to execute a function different from that of the first device; and a controller configured to drive the second device without driving the first device when the magnetic field is applied to the magnetic switch.

The above and other features, advantages and technical and industrial significance of this disclosure will be better understood by reading the following detailed description of presently preferred embodiments of the disclosure, when considered in connection with the accompanying drawings.

### BRIEF DESCRIPTION OF THE DRAWINGS

FIG. 1 is a schematic diagram illustrating an outline of a configuration of a capsule endoscope system according to a first embodiment of the disclosure;

FIG. 2 is a block diagram illustrating a functional configuration of the capsule endoscope according to the first embodiment of the disclosure;

FIG. 3 is a flowchart illustrating an outline of a process executed by the capsule endoscope according to the first embodiment of the disclosure;

FIG. 4 is a diagram illustrating a timing chart of the process executed by the capsule endoscope according to the first embodiment of the disclosure;

FIG. 5 is a block diagram illustrating a functional configuration of a capsule endoscope according to a variation of the first embodiment of the disclosure;

FIG. 6 is a diagram illustrating a timing chart of a process executed by the capsule endoscope according to the variation of the first embodiment of the disclosure;

FIG. 7 is a diagram illustrating a timing chart of a process executed by a capsule endoscope according to a second embodiment of the disclosure;

FIG. 8 is a diagram illustrating a timing chart of a process executed by a capsule endoscope according to a first variation of the second embodiment of the disclosure;

FIG. 9 is a diagram illustrating a timing chart of a process executed by a capsule endoscope according to a second variation of the second embodiment of the disclosure; and

FIG. 10 is a diagram illustrating a timing chart of a process executed by a capsule endoscope according to a third variation of the second embodiment of the disclosure.

### DETAILED DESCRIPTION

Hereinafter, a capsule endoscope system according to an embodiment of the disclosure will be described with reference to the drawings. In the following description, a capsule endoscope orally introduced into a subject and capturing an image is exemplified, but the disclosure is not limited by this embodiment. That is, in the disclosure, it is possible to use various capsule endoscopes, such as a capsule endoscope orally ingested by a subject together with physiological saline solution, water, or the like, and capturing an image of the inside of the body cavity of the subject. In the following description, each figure only schematically illustrates a shape, a size, and a positional relationship to the extent that the contents of the disclosure can be understood. Therefore, the disclosure is not limited exclusively to the shape, the size, and the positional relationship exemplified in each figure. In the drawings, the same parts are denoted by the same reference signs.

### First Embodiment

Configuration of Capsule Endoscope System

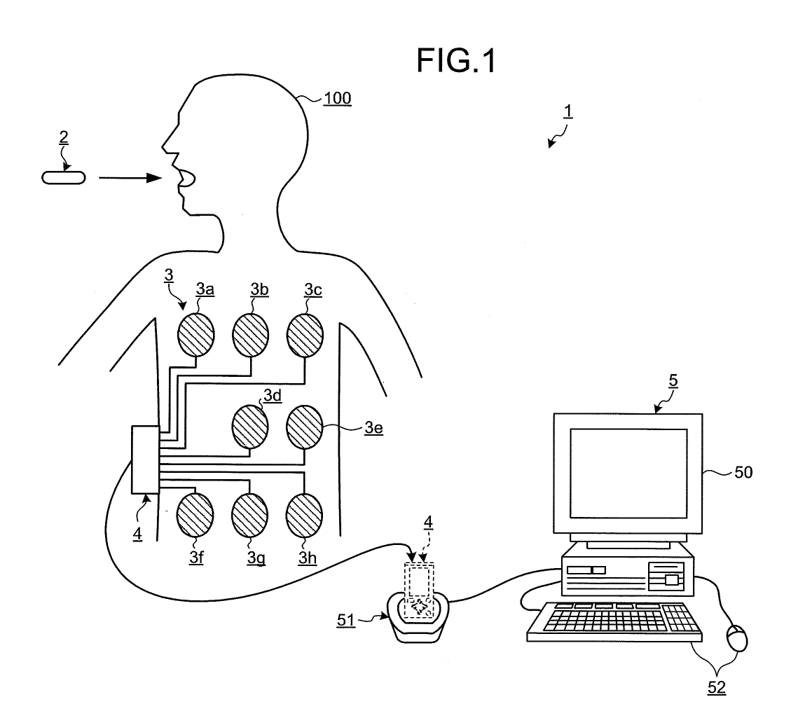

FIG. 1 is a schematic diagram illustrating an outline of a configuration of a capsule endoscope system according to a first embodiment of the disclosure.

The capsule endoscope system 1 illustrated in FIG. 1 includes a capsule endoscope 2, a receiving antenna unit 3, a receiving device 4, and an image processing apparatus 5. The capsule endoscope 2 captures an in-vivo image of a subject 100. The receiving antenna unit 3 receives a radio signal transmitted from the capsule endoscope 2 introduced into the subject 100. To the receiving device 4, the receiving antenna unit 3 is detachably connected and the receiving device 4 performs a predetermined process on the radio signal received by the receiving antenna unit 3 to record or display the radio signal. The image processing apparatus 5 processes and/or displays an image corresponding to image data of the inside of the subject 100 captured by the capsule endoscope 2.

The capsule endoscope 2 has an imaging device which captures an image of the inside of the subject 100 and a wireless communication function for transmitting in-vivo information including image data obtained by capturing an image of the inside of the subject 100 to the receiving antenna unit 3. The capsule endoscope 2 passes through the esophagus in the subject 100 by being swallowed in the subject 100 and moves in the body cavity of the subject 100 by the peristaltic movement of the gastrointestinal lumen. The capsule endoscope 2 successively captures images of the inside of the body cavity of the subject 100 at minute time intervals, for example, 0.5-second intervals (2 fps) while moving in the body cavity of the subject 100, and generates image data of the inside of the subject 100 thus captured and wirelessly transmits sequentially the image data to the receiving antenna unit 3. The detailed configuration of the capsule endoscope 2 will be described later.

The receiving antenna unit 3 includes receiving antennas 3a to 3h. The receiving antennas 3a to 3h receive a radio signal from the capsule endoscope 2 and transmit the radio signal to the receiving device 4. The receiving antennas 3a to 3h are each configured using a loop antenna. Each of the receiving antennas 3a to 3h is attached to a predetermined position on the external surface of the body of the subject 100, for example, at a position corresponding to each organ in the subject 100, which is a passage route of the capsule endoscope 2.

The receiving device 4 records image data of the inside of the subject 100 included in the radio signal received from the capsule endoscope 2 via the receiving antennas 3a to 3h or displays an image corresponding to the image data of the inside of the subject 100. The receiving device 4 records positional information of the capsule endoscope 2 and time information indicating time in association with the radio signal received via the receiving antennas 3a to 3h. While an examination by the capsule endoscope 2 is performed, for example, while the capsule endoscope 2 is introduced from the mouth of the subject 100, passes through the gastrointestinal tract and is excreted from the subject 100, the receiving device 4 is accommodated in a receiving device holder (not illustrated) and is carried by the subject 100. After completion of the examination by the capsule endoscope 2, the receiving device 4 is detached from the subject 100 and is connected to the image processing apparatus 5 in order to transfer image data and the like received from the capsule endoscope 2.

The image processing apparatus 5 includes a display device 50, a cradle 51, and an operation input device 52 such as a keyboard and a mouse. The display device 50 displays an image corresponding to image data of the inside of the subject 100 transferred from the receiving device 4. The cradle 51 reads image data and the like from the receiving device 4. The display device 50 is configured using a display panel employing liquid crystal, organic electro-luminescence (EL), or the like. When the receiving device 4 is attached, the cradle 51 transfers image data, related information such as position information, time information and identification information of the capsule endoscope 2 associated with this image data, from the receiving device 4 to the image processing apparatus 5. The operation input device 52 receives an input by a user. While operating the operation input device 52 and watching the images of the inside of the subject 100 sequentially displayed by the image processing apparatus 5, the user observes biological sites inside the subject 100, for example, the esophagus, the stomach, the small intestine, and the large intestine, and makes a diagnosis on the subject 100.

Configuration of Capsule Endoscope

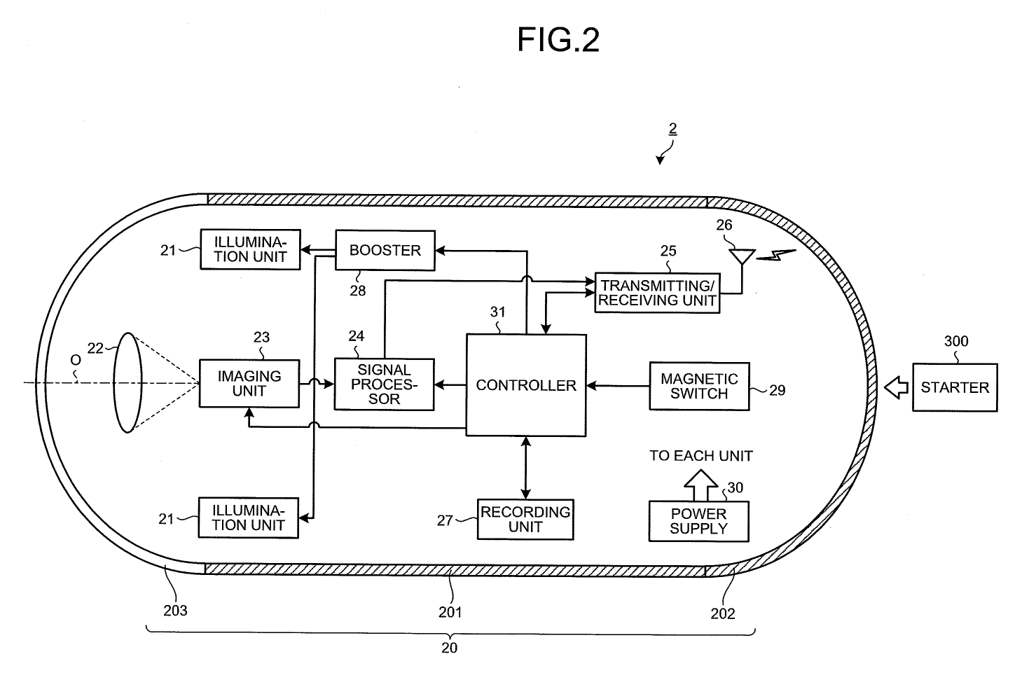

Next, the configuration of the capsule endoscope 2 will be described in detail. FIG. 2 is a block diagram illustrating a functional configuration of the capsule endoscope 2.

The capsule endoscope 2 illustrated in FIG. 2 includes a capsule-shaped casing 20, an illumination unit 21, an optical system 22, an imaging unit 23, a signal processor 24, a transmitting/receiving unit 25, a recording unit 27, a booster 28, a magnetic switch 29, a power supply 30, and a controller 31. The capsule-shaped casing 20 is formed in a size and shape easy to introduce into the gastrointestinal tract of the subject 100. The illumination unit 21 irradiates an imaging view field of the capsule endoscope 2 with illumination light such as white light. The optical system 22 forms a subject image. The imaging unit 23 generates an image signal by receiving the subject image formed by the optical system 22 and performing photoelectric conversion of the subject image. The signal processor 24 performs a predetermined signal process on the image signal generated by the imaging unit 23. The transmitting/receiving unit 25 transmits the image signal input from the signal processor 24 to the outside via an antenna 26 or receives a radio signal from the outside via the antenna 26. The recording unit 27 records various kinds of information of the capsule endoscope 2. The booster 28 boosts a voltage to a predetermined voltage. The magnetic switch 29 detects a magnetic field from the outside. The power supply 30 supplies power to each component unit of the capsule endoscope 2. The controller 31 controls each component unit of the capsule endoscope 2.

The capsule-shaped casing 20 is an outer casing formed in a size and shape which can be introduced into an organ of the subject 100, and is achieved by closing open ends on both sides of a cylindrical casing 201 with dome-shaped casings 202 and 203. The dome-shaped casing 203 is formed using a transparent member capable of transmitting the illumination light with which the illumination unit 21 performs irradiation. As illustrated in FIG. 2, the capsule-shaped casing 20 formed by the cylindrical casing 201 and the dome-shaped casings 202 and 203 includes the optical system 22, the imaging unit 23, the illumination unit 21, the signal processor 24, the transmitting/receiving unit 25, the antenna 26, the recording unit 27, the booster 28, the magnetic switch 29, the power supply 30, and the controller 31.

The illumination unit 21 is a device which, under the control of the controller 31, irradiates an area including at least the imaging view field of the capsule endoscope 2 with illumination light such as white light through the dome-shaped casing 203.

The optical system 22 condenses light reflected from a mucosa of the subject 100 onto an imaging surface of the imaging unit 23 to form a subject image. The optical system 22 is a device configured using one or more lenses, for example, a condenser lens or a focus lens.

The imaging unit 23 generates an image signal of the subject image formed by the optical system 22. The imaging unit 23 is a device configured using an image sensor such as a complementary metal oxide semiconductor (CMOS) or a charge coupled device (CCD).

The signal processor 24 performs a predetermined signal process on the image signal input from the imaging unit 23 and outputs the image signal to the transmitting/receiving unit 25. Here, the predetermined image process is a process such as gain adjustment for the image signal. The signal processor 24 is a device configured using an integrated circuit (IC), a large scale integration (LSI), an application specific integrated circuit (ASIC), or the like. These devices have a circuit including at least a coil (inductor).

The transmitting/receiving unit 25 wirelessly transmits sequentially the image signals input from the signal processor 24 to the outside via the antenna 26. Specifically, the transmitting/receiving unit 25 performs a signal process such as modulation on the image signal input from the signal processor 24 to generate a radio signal, and transmits the radio signal to the outside. In addition, the transmitting/receiving unit 25 receives a radio signal transmitted from the outside via the antenna 26, performs a demodulation process or the like on the radio signal and outputs the radio signal to the controller 31. The transmitting/receiving unit 25 is a device configured using a circuit including at least a coil (inductor).

The recording unit 27 is configured using a flash memory, a read only memory (ROM), or the like, and is a device which records various programs executed by the capsule endoscope 2 and information being processed.

Under the control of the controller 31, the booster 28 boosts a voltage of power supplied from the power supply 30 to a predetermined voltage (first voltage), and supplies the power to the illumination unit 21. Specifically, the booster 28 boosts the voltage of the power supplied from the power supply 30 from 3 V to 15 V and supplies the power to the illumination unit 21. The booster 28 is a device configured using a circuit including at least a cored coil (inductor) having a core. In the first embodiment, the booster 28 functions as one of first devices.

The magnetic switch 29 is a device which detects a magnetic field from the outside and outputs a result of the detection to the controller 31. Specifically, the magnetic switch 29 is configured using a reed switch or a micro-electro-mechanical system (MEMS) switch, and performs switching between connection states depending on the magnetic field from the outside. For example, the magnetic switch 29 performs switching from a first connection state (hereinafter referred to as “unconnected state”), which is one state of a connected state and an unconnected state, to a second connection state (hereinafter referred to as “connected state”), which is the other state thereof, depending on a magnetic field from an external starter 300 (magnetic field applying generator).

The power supply 30 is a device including a storage battery such as a button battery or a capacitor and a switch to be switched by a command from the controller 31. The power supply 30 receives a high-frequency signal of a specific pattern to be a command for performing switching, the high-frequency signal being externally applied, for example, via the transmitting/receiving unit 25, and performs switching between ON and OFF states of the power supply by the control of the controller 31 based on the high-frequency signal. In the ON state, the power supply 30 supplies power to each component unit of the capsule endoscope 2, while in the OFF state, the power supply 30 stops supplying power to each component unit of the capsule endoscope 2.

The controller 31 is a device configured using a central processing unit (CPU) or the like. The controller 31 controls driving of each of component units of the capsule endoscope 2 and controls input and output of signals between each of these component units. For example, each time the imaging unit 23 generates an image signal, the controller 31 causes the signal processor 24 to perform a signal process on the image signal and to wirelessly transmit the image signal to the transmitting/receiving unit 25. When a magnetic field is applied from the starter 300 and the state of the magnetic switch 29 is switched from the unconnected state to the connected state, the controller 31 drives each device of the capsule endoscope 2 not including an inductor such as the imaging unit 23 (a device not using a coil), and thereafter, when the magnetic field ceases to be applied from the starter 300, and the state of the magnetic switch 29 is switched from the connected state to the unconnected state, the controller 31 drives each device including an inductor such as the booster 28 (a device using a coil).

Process of Capsule Endoscope

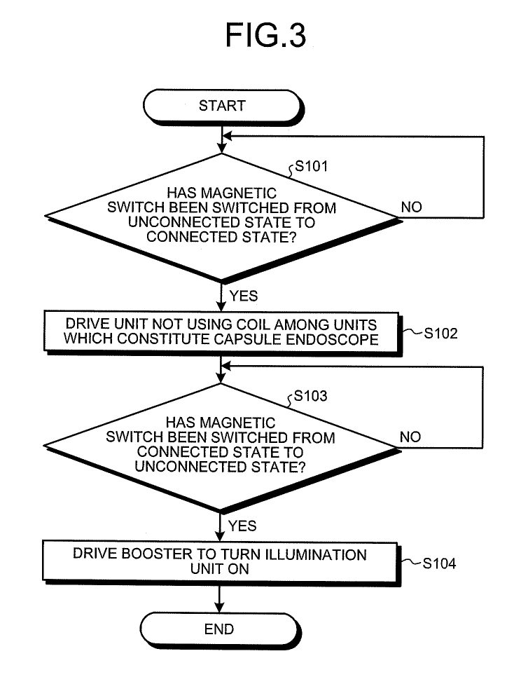

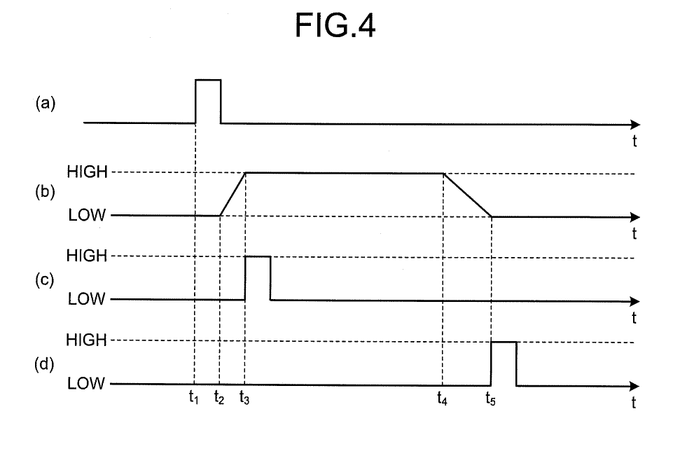

Next, a process executed by the capsule endoscope 2 will be described. FIG. 3 is a flowchart illustrating an outline of a process executed by the capsule endoscope 2, which is a flowchart executed when a magnetic field is applied from the external starter 300. FIG. 4 is a diagram illustrating a timing chart of the process executed by the capsule endoscope 2. In FIG. 4, (a) of FIG. 4 illustrates application timing of the magnetic field by the external starter 300, (b) of FIG. 4 illustrates timing at which the magnetic switch 29 is switched, (c) of FIG. 4 illustrates drive timing of a unit not using a coil among units which constitute the capsule endoscope 2, and (d) of FIG. 4 illustrates drive timing of the booster 28. In (a) of FIG. 4, the rise and fall of the application of the magnetic field schematically illustrate the number of times the magnetic field is applied by the external starter 300, and are merely an example.

As illustrated in FIG. 3, first, when the state of the magnetic switch 29 is switched from the unconnected state to the connected state by application of the magnetic field from the external starter 300 (Step S101: Yes), the controller 31 drives a unit not using a coil among units which constitute the capsule endoscope 2 (Step S102). Specifically, as illustrated in FIG. 4, regarding the controller 31, in response to the application of the magnetic field from the external starter 300 (time t₁), the magnetic switch 29 starts switching from the unconnected state (Low) to the connected state (High) (time t₂), and thereafter, when the magnetic switch 29 is switched from the unconnected state to the connected state (time t₃), the controller 31 drives a component not using a coil, for example, the imaging unit 23, among units which constitute the capsule endoscope 2 (time t₃).

Subsequently, when the external starter 300 moves away from the capsule endoscope 2 and thereby the state of the magnetic switch 29 is switched from the connected state to the unconnected state (Step S103: Yes), the controller 31 drives and causes the booster 28 to turn the illumination unit 21 on (Step S104). Specifically, as illustrated in FIG. 4, when the influence of the magnetic field disappears by the starter 300 moving away from the capsule endoscope 2, the magnetic switch 29 starts switching from the connected state to the unconnected state (time t₄). Thereafter, when the state of the magnetic switch 29 is switched from the connected state to the unconnected state (time t₅), the controller 31 drives the booster 28 (time t₅). Consequently, the booster 28 can be driven in a state in which the influence of the magnetic field from the external starter 300 has disappeared. As a result, since there is no decrease in inductance, the booster 28 can exhibit its original function and can boost the voltage of the power supplied from the power supply 30 to the predetermined voltage. After Step S104, the capsule endoscope 2 terminates the present process.

When the magnetic field is not applied from the external starter 300 and the magnetic switch 29 is not switched from the unconnected state to the connected state in Step S101 (Step S101: No), the capsule endoscope 2 continues this determination until the magnetic field is applied from the external starter 300.

When the state of the magnetic switch 29 is not switched from the connected state to the unconnected state in Step S103 (Step S103: No), the capsule endoscope 2 continues this determination in Step S103 until the magnetic switch 29 is switched to the unconnected state.

According to the first embodiment of the disclosure described above, when a magnetic field is applied while the state of the magnetic switch 29 is the unconnected state and the state of the magnetic switch 29 is switched from the unconnected state to the connected state, the controller 31 drives a device not using a coil such as the imaging unit 23, and thereafter, when the magnetic field ceases to be applied and the state of the magnetic switch 29 is switched from the connected state to the unconnected state, the controller 31 drives a device using a coil such as the booster 28. As a result, it is possible to reliably drive the device using a coil such as the booster 28.

In the first embodiment of the disclosure, when the state of the magnetic switch 29 is switched from the unconnected state to the connected state, the controller 31 drives a device not using a coil such as the imaging unit 23, and thereafter, when the state of the magnetic switch 29 is switched from the connected state to the unconnected state, the controller 31 drives a device using a coil such as the booster 28. However, the disclosure can be applied to any of, for example, a device having a circuit including an inductor (coil), for example, a stepping-down unit (not illustrated) which steps down a voltage of power supplied from the booster 28 to a voltage (second voltage) different from that of the booster 28, the signal processor 24, and the transmitting/receiving unit 25. That is, when the state of the magnetic switch 29 is switched from the unconnected state to the connected state, the controller 31 drives the device not using a coil such as the imaging unit 23, and thereafter, when the state of the magnetic switch 29 is switched from the connected state to the unconnected state, the controller 31 drives any one of the devices using a coil such as the booster 28, the stepping-down unit, the signal processor 24, and the transmitting/receiving unit 25. Consequently, even when a magnetic field is externally applied, it is possible to prevent the device having an inductor from stopping.

Variation of First Embodiment

Next, a variation of the first embodiment of the disclosure will be described. In the above-described first embodiment, the state of the magnetic switch is switched from the connected state to the unconnected state after the influence of the magnetic field by the starter has disappeared. However, in the variation of the first embodiment, a detector which detects the external starter 300 (magnetic field applying generator) is provided and the magnetic switch 29 is switched depending on a detection result of the detector. Therefore, a capsule endoscope according to the variation of the first embodiment will be described below. The same components as those of the capsule endoscope 2 according to the first embodiment described above are denoted by the same reference signs, and description thereof will be omitted.

Configuration of Capsule Endoscope

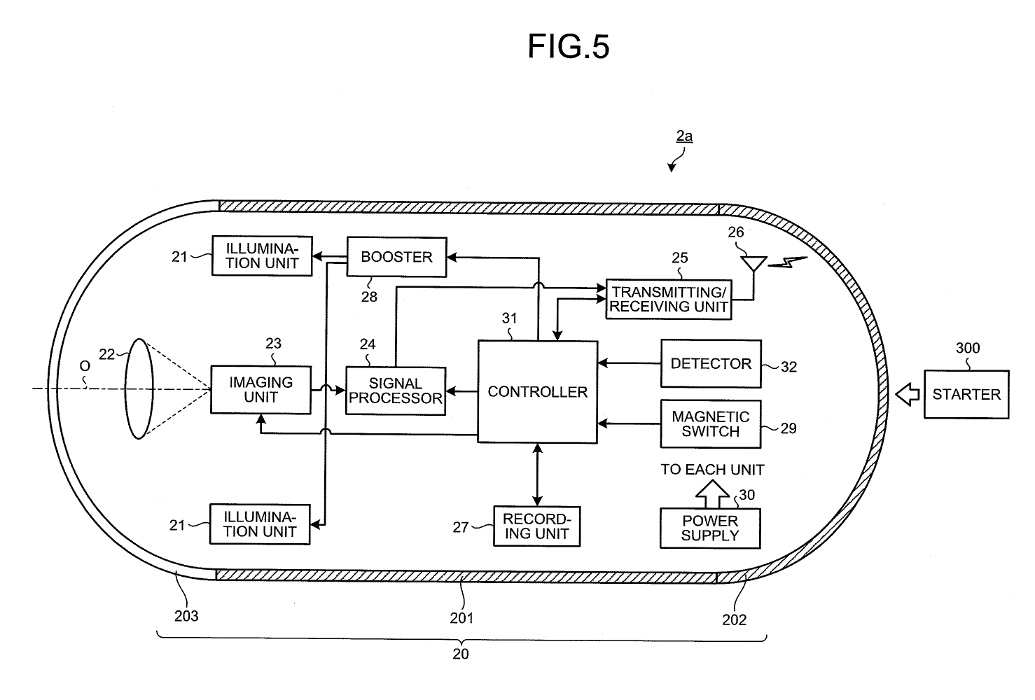

FIG. 5 is a block diagram illustrating a functional configuration of the capsule endoscope according to the variation of the first embodiment of the disclosure. The capsule endoscope 2a illustrated in FIG. 5 further includes a detector 32 in addition to the configuration of the capsule endoscope 2 according to the first embodiment described above.

The detector 32 detects that the external starter 300 has approached the capsule endoscope 2a and outputs a result of the detection to the controller 31. The detector 32 is a device configured using a non-contact-type physical-quantity sensor such as an ultrasonic sensor or an acceleration sensor, or a magnetic sensor. Specifically, when the acceleration sensor is used as the physical-quantity sensor, the detector 32 detects the presence or absence of contact of the starter 300 with the capsule endoscope 2a; when the ultrasonic sensor is used as the physical-quantity sensor, the detector 32 detects the presence of the starter 300 by detecting the distance between the capsule endoscope 2a and the starter 300; and when the magnetic sensor is used as the physical-quantity sensor, the detector 32 detects a magnetic field radiated by the starter 300. In the following description, the detector 32 will be described by taking as an example a device that detects a magnetic field radiated by the starter 300.

Process of Capsule Endoscope

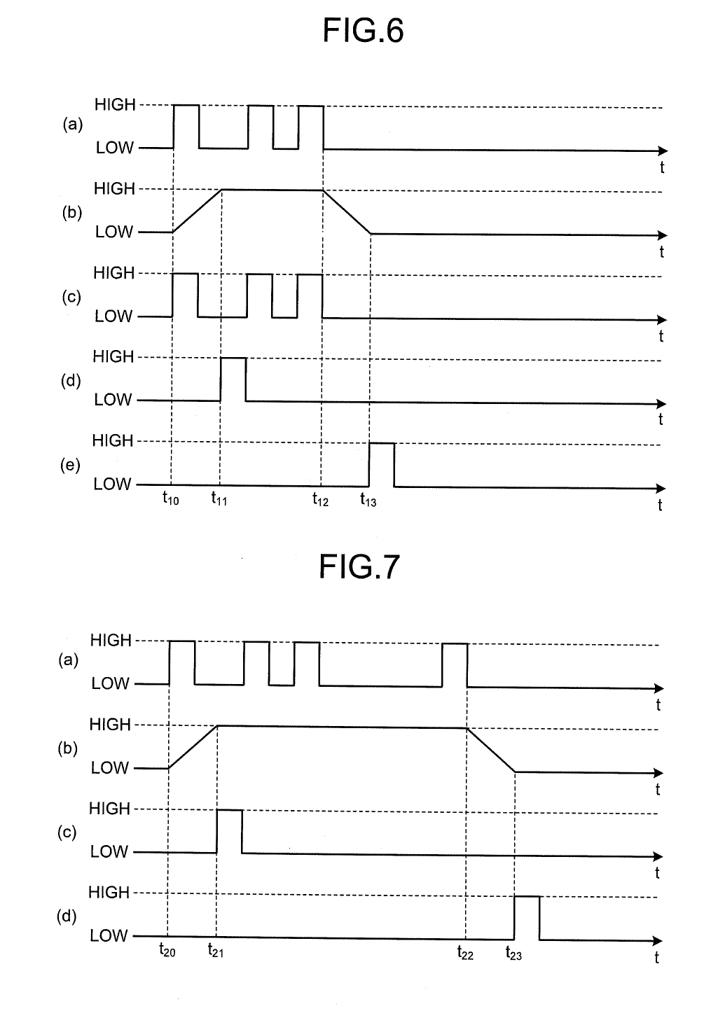

Next, a process executed by the capsule endoscope 2a will be described. FIG. 6 is a diagram illustrating a timing chart of the process executed by the capsule endoscope 2a. In FIG. 6, (a) of FIG. 6 illustrates application timing of the magnetic field by the external starter 300, (b) of FIG. 6 illustrates timing at which the magnetic switch 29 is switched, (c) of FIG. 6 illustrates detection timing of the detector 32, (d) of FIG. 6 illustrates drive timing of a unit not using a coil among units which constitute the capsule endoscope 2a, and (e) of FIG. 6 illustrates drive timing of the booster 28. In (a) of FIG. 6, the rise and fall of the application of the magnetic field schematically illustrate the number of times the magnetic field is applied by the external starter 300, and are merely an example.

As illustrated in FIG. 6, when the external starter 300 approaches the capsule endoscope 2a and the magnetic field is applied from the external starter 300 (time t₁₀), and thereby the state of the magnetic switch 29 is switched from the unconnected state (Low) to the connected state (High) (time t₁₁), the controller 31 drives a unit not using a coil, for example, the imaging unit 23, among units which constitute the capsule endoscope 2a (time t₁₁).

Subsequently, after a predetermined period of time (for example, one second) from timing at which the detector 32 ceases to detect the magnetic field radiated from the external starter 300 (time t₁₂), the magnetic switch 29 is switched from the connected state to the unconnected state (time t₁₃). Thereafter, the controller 31 drives and causes the booster 28 to turn the illumination unit 21 on (time t₁₃). Consequently, since the booster 28 is driven in a state where the influence of the magnetic field radiated from the starter 300 has disappeared, the voltage of the power supplied from the power supply 30 can be boosted to a predetermined voltage without lowering the inductance of the booster 28.

According to the variation of the first embodiment of the disclosure described above, the controller 31 drives a device using a coil such as the booster 28 depending on the detection result of the detector 32 after the state of the magnetic switch 29 is switched from the connected state to the unconnected state, and consequently, even when the magnetic field is applied a plurality of times from the external starter 300, the device using a coil such as the booster 28 can be driven in a state in which the influence of the magnetic field radiated from the starter 300 has disappeared. As a result, it is possible to reliably drive the device using a coil such as the booster 28.

### Second Embodiment

Next, a second embodiment of the disclosure will be described. The second embodiment of the disclosure has the same configuration as the capsule endoscope 2 according to the first embodiment described above, and differs only in a process to be executed. Hereinafter, the process of the capsule endoscope according to the second embodiment will be described. The same components as those of the capsule endoscope 2 according to the first embodiment described above are denoted by the same reference signs, and description thereof will be omitted.

Process of Capsule Endoscope

FIG. 7 is a diagram illustrating a timing chart of the process executed by the capsule endoscope 2 according to the second embodiment. In FIG. 7, (a) of FIG. 7 illustrates application timing of a magnetic field by an external starter 300, (b) of FIG. 7 illustrates timing at which a magnetic switch 29 is switched, (c) of FIG. 7 illustrates drive timing of a unit not using a coil among units which constitute the capsule endoscope 2, and (d) of FIG. 7 illustrates drive timing of a booster 28. In (a) of FIG. 7, the rise and fall of the application of the magnetic field schematically illustrate the number of times the magnetic field is applied by the external starter 300, and are merely an example.

As illustrated in FIG. 7, first, in response to the application of the magnetic field from the external starter 300 (time t₂₀), the magnetic switch 29 starts switching from an unconnected state (Low) to a connected state (High) (time t₂₀).

Thereafter, when the state of the magnetic switch 29 is switched from the unconnected state to the connected state (time t₂₁), a controller 31 drives a unit not using a coil, for example, an imaging unit 23, among units which constitute the capsule endoscope 2 (time t₂₁).

Subsequently, after a predetermined period of time, for example, one second, from a state where the connected state is established (time t₂₁), the magnetic switch 29 starts switching the state from the connected state to the unconnected state (time t₂₂). Thereafter, after the influence of the magnetic field on the capsule endoscope 2 disappears and the state of the magnetic switch 29 is switched from the connected state to the unconnected state (time t₂₃), the controller 31 drives a device using a coil such as the booster 28 (time t₂₃). Consequently, even when the magnetic field is applied a plurality of times from the external starter 300, it is possible to delay activation timing of the booster 28. As a result, since there is no decrease in inductance, the booster 28 can exhibit its original function and can boost a voltage of power supplied from a power supply 30 to a predetermined voltage.

According to the second embodiment of the disclosure described above, when a magnetic field is applied while the state of the magnetic switch 29 is the unconnected state, and the state of the magnetic switch 29 is switched from the unconnected state to the connected state, the controller 31 drives a device not using a coil such as the imaging unit 23, and thereafter, when the magnetic field ceases to be applied and the state of the magnetic switch 29 is switched from the connected state to the unconnected state, the controller 31 drives a device using a coil such as the booster 28. As a result, it is possible to reliably drive the device using a coil such as the booster 28.

In addition, according to the second embodiment of the disclosure, the magnetic switch 29 switches the state from the connected state to the unconnected state after a predetermined period of time has elapsed since the application of the magnetic field from the starter 300, and thereby even when the magnetic field is applied a plurality of times from the external starter 300 during the period of switching from the connected state to the unconnected state, the controller 31 can delay the activation timing of a device using a coil such as the booster 28. As a result, it is possible to reliably drive the device using a coil such as the booster 28.

In the second embodiment of the disclosure, when the state of the magnetic switch 29 is switched from the unconnected state to the connected state, the controller 31 drives a device not using a coil such as the imaging unit 23, and thereafter, when the state of the magnetic switch 29 is switched from the connected state to the unconnected state, the controller 31 drives a device using a coil such as the booster 28. However, the disclosure can be applied to any of, for example, a device having a circuit including an inductor (coil), for example, a stepping-down unit (not illustrated) which steps down a voltage of power supplied from the booster 28 to a voltage (second voltage) different from that of the booster 28, a signal processor 24, and a transmitting/receiving unit 25. That is, when the state of the magnetic switch 29 is switched from the unconnected state to the connected state, the controller 31 drives the device not using a coil such as the imaging unit 23, and thereafter, when the state of the magnetic switch 29 is switched from the connected state to the unconnected state, the controller 31 drives any one of the devices using a coil such as the booster 28, the stepping-down unit, the signal processor 24, and the transmitting/receiving unit 25. Consequently, even when a magnetic field is externally applied, it is possible to prevent the device having an inductor from stopping.

In the second embodiment of the disclosure, when the number of times the state of the magnetic switch 29 was switched from the unconnected state to the connected state reaches a predetermined number, for example, three, the controller 31 may drive a device using a coil such as the booster 28. As a matter of course, the controller 31 may drive the device using a coil such as the booster 28 after a predetermined period of time (for example, one second to three seconds) has elapsed since the number of times the state of the magnetic switch 29 was switched from the unconnected state to the connected state reached the predetermined number. Consequently, it is possible to more reliably prevent the influence of the magnetic field of the starter 300.

First Variation of Second Embodiment

Next, a first variation of the second embodiment of the disclosure will be described. The first variation of the second embodiment of the disclosure has the same configuration as the capsule endoscope 2 according to the second embodiment described above, and differs only in a process to be executed. Hereinafter, the process of the capsule endoscope according to the first variation of the second embodiment will be described. The same components as those of the capsule endoscope 2 according to the second embodiment described above are denoted by the same reference signs, and description thereof will be omitted.

Process of Capsule Endoscope

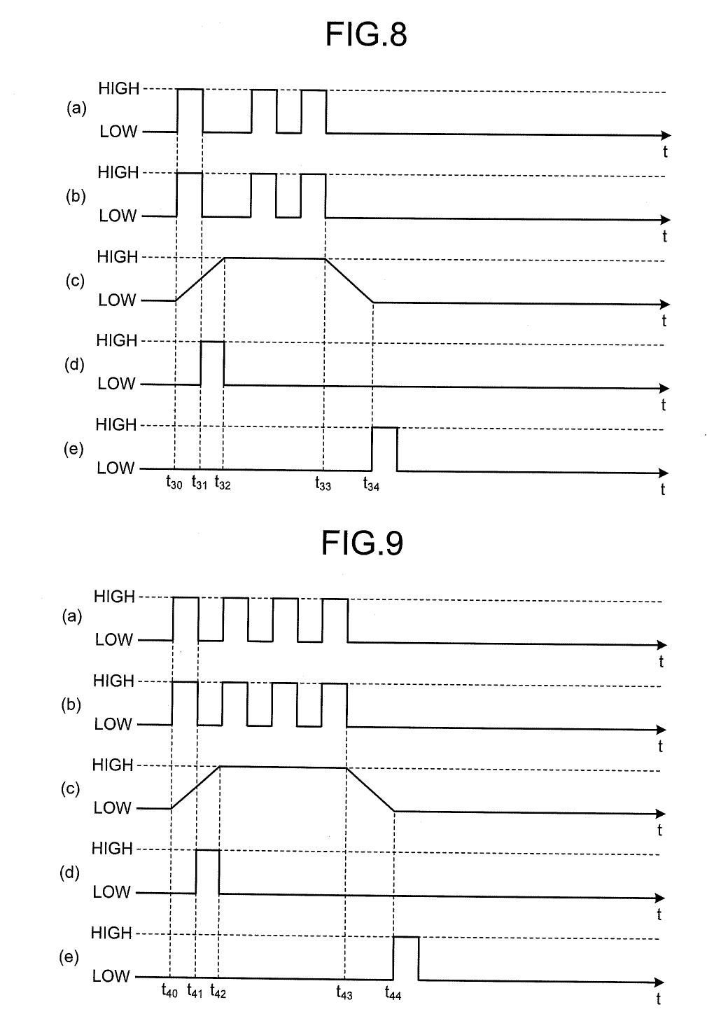

FIG. 8 is a diagram illustrating a timing chart of a process executed by the capsule endoscope 2 according to the first variation of the second embodiment of the disclosure. In FIG. 8, (a) of FIG. 8 illustrates application timing of the magnetic field by the external starter 300, (b) of FIG. 8 illustrates timing at which the state of the magnetic switch 29 is switched, (c) of FIG. 8 illustrates setting and canceling timings of a flag as information indicating that the state of the magnetic switch 29 recorded by a recording unit 27 is the connected state, (d) of FIG. 8 illustrates drive timing of a unit not using a coil among units which constitute the capsule endoscope 2, and (e) of FIG. 8 illustrates drive timing of the booster 28.

As illustrated in FIG. 8, when the magnetic field is applied from the external starter 300 (time t₃₀) and the state of the magnetic switch 29 is switched from the unconnected state to the connected state (time t₃₀), the controller 31 drives a unit not using a coil, for example, the imaging unit 23, among units which constitute the capsule endoscope 2 (time t₃₁), and sets (records) a flag as information indicating that the state of the magnetic switch 29 has been switched to the connected state in the recording unit 27 (time t₃₂).

Subsequently, each time the magnetic field is applied from the external starter 300, the magnetic switch 29 switches the state between the unconnected state and the connected state. In that case, the controller 31 does not drive the device using a coil such as the booster 28 until a predetermined period of time (for example, one to two seconds) elapses from the timing at which the flag is set while the magnetic switch 29 is in the connected state.

Thereafter, the controller 31 cancels (deletes) the flag after a predetermined period of time (for example, one second to two seconds) has elapsed (time t₃₃) and drives the device using a coil such as the booster 28 (time t₃₄).

According to the first variation of the second embodiment of the disclosure described above, the controller 31 drives a device using a coil such as the booster 28 after the predetermined period of time has elapsed since the recording timing of the flag as the information indicating that the state of the magnetic switch 29 has been switched to the connected state, the flag being recorded in the recording unit 27, and consequently, even when the magnetic field is applied a plurality of times from the external starter 300, the drive timing can be delayed for the device using a coil such as the booster 28. As a result, it is possible to reliably drive the device using a coil such as the booster 28.

In the first variation of the second embodiment of the disclosure, the detector 32 of the variation of the above-described first embodiment may be provided. In that case, the controller 31 drives a device using a coil such as the booster 28 depending on the detection result of the detector 32, after the state of the magnetic switch 29 is switched from the connected state to the unconnected state, and consequently, even when the magnetic field is applied a plurality of times from the external starter 300, the drive timing can be delayed for the device using a coil such as the booster 28. As a result, it is possible to reliably drive the device using a coil such as the booster 28.

Second Variation of Second Embodiment

Next, a second variation of the second embodiment of the disclosure will be described. The second variation of the second embodiment of the disclosure has the same configuration as the capsule endoscope 2 according to the second embodiment described above, and differs only in a process to be executed. Hereinafter, the process of the capsule endoscope according to the second variation of the second embodiment will be described. The same components as those of the capsule endoscope 2 according to the second embodiment described above are denoted by the same reference signs, and description thereof will be omitted.

Process of Capsule Endoscope

FIG. 9 is a diagram illustrating a timing chart of the process executed by the capsule endoscope 2 according to the second variation of the second embodiment of the disclosure. (a) of FIG. 9 illustrates application timing of the magnetic field by the external starter 300, (b) of FIG. 9 illustrates timing at which the state of the magnetic switch 29 is switched, (c) of FIG. 9 illustrates setting and canceling timings of a flag as information indicating that the state of the magnetic switch 29 recorded by the recording unit 27 is the connected state, (d) of FIG. 9 illustrates drive timing of a unit not using a coil among units which constitute the capsule endoscope 2, and (e) of FIG. 9 illustrates drive timing of the booster 28.

As illustrated in FIG. 9, when the magnetic field is applied from the external starter 300 (time t₄₀) and the state of the magnetic switch 29 is switched from the unconnected state to the connected state (time t₄₀), the controller 31 drives a unit not using a coil, for example, the imaging unit 23, among units which constitute the capsule endoscope 2 (time t₄₁), and, in the recording unit 27, sets a flag as information indicating that the state of the magnetic switch 29 has been switched to the connected state in the recording unit 27 (time t₄₂).

Subsequently, each time the magnetic field is applied from the external starter 300, the magnetic switch 29 switches the state between the unconnected state and the connected state. In that case, when the number of times the state of the magnetic switch 29 was switched from the unconnected state to the connected state reaches a predetermined number (for example, four) after the capsule endoscope 2 was brought into the connected state (the timing at which the magnetic field was first applied from the starter 300 (time t₄₀)), the controller 31 cancels the flag of the recording unit 27 (time t₄₃).

Thereafter, at the timing at which the flag is canceled (time t₄₃), the controller 31 drives the device using a coil such as the booster 28 (time t₄₄).

According to the second variation of the second embodiment of the disclosure described above, when the number of times the state of the magnetic switch 29 was switched from the unconnected state to the connected state reaches a predetermined number, for example, four, the controller 31 drives a device using a coil such as the booster 28, and consequently, even when the magnetic field is applied a plurality of times from the external starter 300, the activation timing can be delayed for the device using a coil such as the booster 28. As a result, it is possible to reliably drive the device using a coil such as the booster 28.

Third Variation of Second Embodiment

Next, a third variation of the second embodiment of the disclosure will be described. The third variation of the second embodiment of the disclosure has the same configuration as the capsule endoscope 2 according to the second embodiment described above, and differs only in a process to be executed. Hereinafter, the process of the capsule endoscope according to the third variation of the second embodiment will be described. The same components as those of the capsule endoscope 2 according to the second embodiment described above are denoted by the same reference signs, and description thereof will be omitted.

Process of Capsule Endoscope

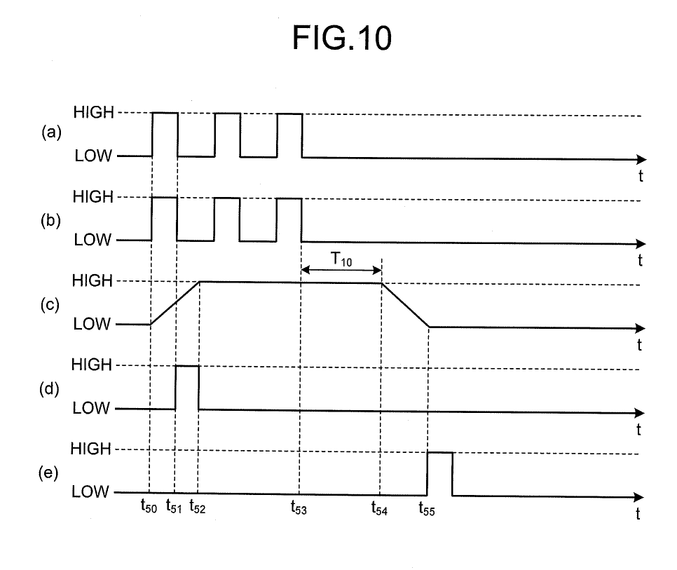

FIG. 10 is a diagram illustrating a timing chart of the process executed by the capsule endoscope 2 according to the third variation of the second embodiment of the disclosure. (a) of FIG. 10 illustrates application timing of the magnetic field by the external starter 300, (b) of FIG. 10 illustrates timing at which the state of the magnetic switch 29 is switched, (c) of FIG. 10 illustrates setting and canceling timings of a flag as information indicating that the state of the magnetic switch 29 recorded by the recording unit 27 is the connected state, (d) of FIG. 10 illustrates drive timing of a unit not using a coil among units which constitute the capsule endoscope 2, and (e) of FIG. 10 illustrates drive timing of the booster 28.

As illustrated in FIG. 10, when the starter 300 approaches the capsule endoscope 2, the magnetic field is applied from the starter 300 (time t₅₀), and the state of the magnetic switch 29 is switched from the unconnected state to the connected state (time t₅₀), the controller 31 drives a unit not using a coil, for example, the imaging unit 23, among units which constitute the capsule endoscope 2 (time t₅₁), and sets a flag indicating that the state of the magnetic switch 29 has been switched to the connected state in the recording unit 27 (time t₅₂).

Subsequently, when a predetermined period of time of T₁₀ (for example, one second to three seconds) has elapsed since the timing at which the state of the magnetic switch 29 was switched from the connected state to the unconnected state (time t₅₃), the controller 31 cancels the flag (time t₅₄).

Thereafter, at the timing at which the flag is canceled (time t₅₅), the controller 31 drives the device using a coil such as the booster 28 (time t₅₅).

According to the third variation of the second embodiment of the disclosure described above, after the predetermined period of time (one second to three seconds) has elapsed since the state of the magnetic switch 29 was switched from the unconnected state to the connected state, the controller 31 drives a device using a coil such as the booster 28 after the switching of the state of the magnetic switch 29 to the unconnected state, and consequently, even when the magnetic field is applied a plurality of times from the external starter 300, the activation timing can be delayed for the device using a coil such as the booster 28. As a result, it is possible to reliably drive the device using a coil such as the booster 28.

According to the capsule endoscope of the disclosure, even when a strong magnetic field is externally applied, a function of a circuit having an inductor can be sufficiently exerted.

The disclosure is not limited to the above-described embodiments, and it goes without saying that various variations and applications are possible within the scope of the gist of the disclosure. For example, the disclosure can be applied to an imaging device, a medical device, or the like that can be inserted into a subject other than the capsule endoscope used in the description of the disclosure.

In addition, in the description of each operation flowchart described above in the specification, the operations have been described using “first”, “next”, “subsequently”, “thereafter”, and the like, for the sake of convenience. However, it does not mean that it is essential to carry out the operations in this order.

Each of the processing methods performed by the capsule endoscope in the above-described embodiments, that is, the process illustrated in each flowchart, may be stored as a program which can be executed by a controller such as a CPU. In addition, such a program can be distributed after stored in a storage medium of an external storage device such as a memory card (ROM card, RAM card, etc.), a magnetic disk, a hard disk, an optical disk (CD-ROM, DVD, etc.), or a semiconductor memory. Then, the controller such as a CPU reads the program stored in the storage medium of the external storage device, and the operations are controlled by the read program so that the above-described processes can be executed.

The disclosure is not limited to the above-described embodiments and variations as they are. In the implementation stage, the constituent elements can be modified and embodied without departing from the gist of the disclosure. In addition, various disclosures can be formed by appropriately combining a plurality of constituent elements disclosed in the above embodiments. For example, some constituent elements may be deleted from all constituent elements described in the embodiments and the variations described above. Furthermore, the constituent elements described in the respective embodiments and the variations may be appropriately combined.

A term described at least once in the specification or the drawings together with a different term which is broader than or equivalent to the term can be replaced with the different term at any point in the specification or the drawings. As described above, various modifications and applications are possible without departing from the gist of the disclosure.

What is claimed is:

1. A capsule endoscope configured to be introduced into a subject, the capsule endoscope comprising:
a magnetic switch configured to operate depending on a magnetic field externally applied;
a first device including an inductor and configured to execute a predetermined function;
a second device including no inductor and configured to execute a function different from that of the first device; and
a controller configured to drive the second device without driving the first device when the magnetic field is applied to the magnetic switch.

2. The capsule endoscope according to claim 1, wherein the controller is configured to drive the first device when the magnetic field ceases to be applied to the magnetic switch.

3. The capsule endoscope according to claim 1, wherein a connection state of the magnetic switch is switched after a predetermined period of time has elapsed since an application of the magnetic field.

4. The capsule endoscope according to claim 1, wherein the controller is configured to drive the first device when the number of times the connection state of the magnetic switch was switched after the driving of the second device reaches a preset number.

5. The capsule endoscope according to claim 2, further comprising a memory configured to record information indicating that the connection state of the magnetic switch has been switched, wherein
in a case where the connection state of the magnetic switch has been switched, the controller is configured to drive the first device when a predetermined period of time has elapsed since timing at which the memory started recording the information.

6. The capsule endoscope according to claim 2, further comprising a memory configured to record information indicating that the connection state of the magnetic switch has been switched, wherein
in a case where the connection state of the magnetic switch has been switched, the controller is configured to delete the information from the memory, and drive the first device when a predetermined period of time has elapsed since timing of deleting the information.

7. The capsule endoscope according to claim 2, further comprising a detector configured to detect an approach of a magnetic field applying generator configured to apply the magnetic field to the magnetic switch, wherein
in a case where the connection state of the magnetic switch has been switched, the controller is configured to drive the first device when the detector has ceased to detect the approach of the magnetic field applying generator.

8. The capsule endoscope according to claim 1, wherein
the second device includes at least an image sensor configured to perform capturing of an inside of the subject to generate an image, and
the first device is
at least one of a booster configured to boost a voltage of power supplied from a power supply to a predetermined first voltage, a stepping-down circuit configured to step down a voltage of externally supplied power to a second voltage different from the first voltage, a transmitting and receiving circuit configured to transmit the image to an external device or receive information from the external device, and a signal processor configured to perform a signal process on the image generated by the image sensor.

9. The capsule endoscope according to claim 1, wherein
the magnetic switch is switched to a connected state when the magnetic field is applied to the magnetic switch, and is switched to an unconnected state when the magnetic field ceases to be applied to the magnetic switch, and
the controller is configured to drive the second device without driving the first device when the magnetic field is applied to the magnetic switch and the magnetic switch is switched from the unconnected state to the connected state, and the controller is configured to drive the first device when the magnetic field ceases to be applied to the magnetic switch and the magnetic switch is switched from the connected state to the unconnected state.
